(19) United States
(12) Patent Application Publication
(10) Pub. No.: US 2005/0102039 A1
(43) Pub. Date: May 12, 2005

(54) Hucklebone supporting-type artificial leg

(76) Inventor: Isao Ikeda, Shioya-gun (JP)

Correspondence Address: WALLACE G. WALTER, 5726 CLARENCE AVE, ALEXANDRIA, VA, 22311-1008, US

(21) Appl. No.: 10/983,895

(22) Filed: Nov. 9, 2004

(30) Foreign Application Priority Data
Nov. 12, 2003 (JP) 2003-382015

(51) Int. Cl. A61F 2/62; A61F 2/78

(52) U.S. Cl. 623/32; 623/38

(57) ABSTRACT

This invention enables a smooth walking action to be carried out even in the case that a length of the thigh portion left in the damaged or lost lower leg is short or in the case that the lower leg is cut from the crotch articulation part and the thigh is lost. A hucklebone supporting-type artificial leg comprised of a saddle, a shaft suspended down at the lower surface of the saddle and having a substantial same length as that of a lower leg and a foot fixed to the lower end of the shaft, wherein the artificial leg comprises a front belt supporting unit attached at a front end of the saddle in such a way that it can be turned in a forward, upward and downward direction; a rear belt supporting unit attached at a rear end of the saddle in such a way that it can be turned in a rearward, upward and downward direction; and waist belts connected to each of the upper ends of the front and rear belt supporting units and installed around the waist of an artificial leg wearing person in such a way that the belts can be fastened to the waist and engaged with or disengaged from the waist. Further, there are provided shoulder hanging bands having ends connected to each of upper ends of the front and rear belt supporting units and installed in such a way that they can be fastened to and removably set to the shoulders of the artificial leg wearing person.

### BACKGROUND OF THE INVENTION

1. Field of the Invention

This invention relates to the hucklebone supporting-type artificial leg so as to support a physical weight while arranging a saddle at the upper end of the artificial leg and abutting it against the crotch.

2. Description of the Related Art

As the hucklebone supporting-type artificial leg for use in supporting a physical weight by the hucklebone while arranging a saddle against the upper end of the artificial leg and abutting it against the crotch and at the same time for fixing the thigh left at the lost lower leg, “the artificial leg” described in Patent Document 1 or “the saddle type crutches” described in Patent Document 2 is provided. These units enabled a person lost the lower leg or injured due to accident, mine and disease or the like to install the artificial leg and walk at an early stage where the injury portion is not completely healed because the saddle is abutted against the crotch to support a physical weight of the person. In addition, a mass production of these units could be carried out because it is not necessary to make them under an order-made manner while being aligned with the shape of damaged portion in the damaged lower leg as found in the prior art artificial leg and in this way the mass production enabled the artificial leg to be provided in a less expensive price.

[Patent Document 1] Gazette of Design Registration No. 1163891

[Patent Document 2] Gazette of Japanese Patent Laid-Open No. 2002-34717

As described above, the hucklebone supporting-type artificial leg provides a superior feeling in wearing to a person because no load is applied to the extremity end of the damaged or lost lower leg, it does not require any special walking training and the person can walk just after wearing it. However, in the case that the length of the thigh left in the damaged or lost lower leg is short, the artificial leg and the thigh are insufficiently fixed. In addition, in the case that the lower leg is cut at the crotch articulation part, the artificial leg cannot be fixed to the thigh portion. Under these cases, there occurred a problem that the artificial leg is insufficiently fixed to the physical body and the work for pulling up the rear artificial leg and moving it in a forward direction cannot be carried out smoothly during walking.

In addition, in the case that the person runs while the artificial leg is being fixed to the thigh portion of the damaged or lost lower leg, a motion of the foot at the extremity end of the artificial leg causes the foot to be moved forward or rearward like a pendulum around the center of the saddle of the crotch. At this time, a swinging angle of the foot in a forward and upward direction and a swinging angle of the foot in a rearward and upward direction become substantially equal to each other. In turn, as to a motion of the thigh portion of a normal lower leg not wearing the artificial leg, the thigh portion shows a large swinging angle in a forward and upward direction due to the presence of the knee joint and correspondingly its rearward-swinging angle becomes low. In this way, as compared with the thigh portion of the normal leg, a motion range of the artificial leg is eccentrically displaced in a rearward direction, resulting in that the thigh portion of the damaged or lost leg is forced to be inclined in a rearward direction which does not occur during running with the normal legs. As a result, in the case that the thigh portion is left in a rearward direction during walking and the artificial leg is inclined in a rearward direction, the pelvis is simultaneously inclined and the upper half body is inclined forwardly or twisted to cause a walking attitude to become disturbed.

Additionally, looking at the attitude from a forward direction under a state in which the hucklebone supporting-type artificial leg is worn by the person indicates that the normal lower leg supports a physical weight applied to the lower leg by the crotch articulation at the end portion of the pelvis, and the physical weight applied to the lower leg is supported at the hucklebone portion of the crotch through the saddle because the damaged or lost lower leg wears the artificial leg. As a result, an attitude for supporting the upper physical body shows an unbalanced state at the right half and the left half, the attitude easily becomes a state in which the upper physical body moves to the normal lower leg, and the attitude of the upper physical body during walking is also easily disturbed. In view of the above fact, it is an object of the present invention to provide the hucklebone supporting-type artificial leg capable of making a more smooth action when the artificial leg is moved forward or rearward during walking and further reducing a disturbed attitude. Further, it is an object of the present invention to provide a hucklebone supporting-type artificial leg reducing more an unbalanced state of right half and left half physical weights applied to the artificial leg and improving more a wearing comfortable state.

### SUMMARY OF THE INVENTION

In order to solve the aforesaid problems, the present invention provides a hucklebone supporting-type artificial leg comprising a saddle; a shaft vertically suspended at the i lower surface of the saddle and having a substantial same length as that of the lower leg; and a foot fixed to the lower end of the shaft, wherein there are provided a front belt supporting unit attached at the front end of the saddle in such a way that its forward, upward and downward rotation can be carried out; a rear belt supporting unit attached at the rear end of the saddle in such a way that its rearward, upward and downward rotation can be carried out; and waist belts each of which is connected to each of the upper ends of the front and rear belt supporting units and installed around the waist of an artificial leg wearing person in such a way that they can be fastened and engaged with or disengaged from the waist. In this case, it is preferable to provide a shoulder hanging band of which end portion is connected to each of the upper ends of the front and rear belt supporting units and installed in such a way that it can be fastened or removably installed at the shoulders of the artificial leg wearing person. In addition, it is preferable that the upper portion of the shaft is bent in such a way that it may be offset from a connecting position at the center of the saddle in a slant forward direction of the damaged or lost lower leg. In addition, it is preferable that a connecting portion between the upper end of the shaft and the saddle is of a fastening structure using threads and the offset direction of the upper portion of the shaft can be changed over at a right side and a left side.

Additionally, it is also possible to provide a fastening structure in which an intermediate position in a forward or rearward direction of a lower side ground surface of the foot, i.e. a position offset to one side in a lateral direction is set as a connecting position with the lower end of the shaft, a connecting portion between the lower end of the shaft and the foot is applied as a fastening structure using threads and the offset direction of the foot can be changed over at a right side and a left side, respectively. In addition, it is also possible to provide a thigh pad having a concave surface shape fixed to the upper portion of the shaft in a slant front downward direction so as to cause the thigh portion of the damaged or lost lower leg to be fixed by bands. In addition, it is also possible that a cylinder movable up and down is applied to cover an intermediate portion of the shaft, the shaft in the cylinder placed at the position where the cylinder descends is divided into an upper portion and a lower portion and they are connected by a hinge and the upper portion of the shaft can be inclined under a state in which the cylinder is ascended. In addition, an optional section in the shaft is set as a double-shaft structure and at the same time a plurality of holes in a diameter direction are punched at the double-shaft structure in an appropriate axial spacing, pins are inserted into the holes in such a way that they can be freely pulled out of them to enable a length of the shaft to be adjusted in a stepwise manner.

The present invention can be utilized as an artificial leg in the case that a lower leg is damaged or lost due to accident, mine and diseases or the like, and further the present invention can be used in place of a set of crutches in the case that one leg cannot support the physical weight due to a breakage of bone or injury.

As described above, the present invention enables an installing characteristic of the artificial leg to be improved and a stable walking of the wearing person to be carried out by a method in which each of front and rear belt supporting units is attached to the front end and rear end of the saddle in such a way that they can be turned in an upward or downward direction, waist belts are connected to each of the upper ends of the front and rear belt supporting units and further installed around the waist of the artificial leg wearing person in such a way that they can be fastened and removably set. In addition, the artificial leg wearing characteristic can be further improved and a stable walking of the leg wearing person can be attained by a method wherein the end portions of the shoulder hanging bands are connected to the upper ends of the front and rear belt supporting units, and supported such that the saddle is hung at each of the shoulders of the artificial leg wearing person.

Further, a stable characteristic of the artificial leg supporting a physical weight can be increased and a walking state can be made more smooth by a method wherein the upper portion of the shaft is offset from the central connecting position of the saddle to the slant front side of the damaged or lost lower leg. In addition, the connecting part between the upper end of the shaft and the saddle is of a fastening structure using threads and the offset direction at the upper end of the shaft can be changed over between a right side and a left side to enable the artificial leg for a right foot and the artificial leg for a left foot to be applied in common. Further, it becomes possible to change over the offset direction at the foot part in response to a degree of training in walking after wearing the artificial leg and use the artificial leg separately for each of the right and left feet.

### BRIEF DESCRIPTION OF THE DRAWINGS

FIG. 1 is a front elevational view for showing the hucklebone supporting-type artificial leg of the present invention.

FIG. 2 is a left side elevational view for showing the hucklebone supporting-type artificial leg of the present invention.

FIG. 3 is a right side elevational view for showing the hucklebone supporting-type artificial leg of the present invention.

FIG. 4 is a rear view for showing the hucklebone supporting-type artificial leg of the present invention.

FIG. 5 is a top plan view for showing the hucklebone supporting-type artificial leg of the present invention.

FIG. 6 is a bottom view for showing the hucklebone supporting-type artificial leg of the present invention.

FIG. 7 is a left side elevational view for showing a state in which a shoulder-hanging band and a fastening band for fixing the thigh are removed from FIG. 2.

FIG. 8 is a perspective view for showing a state in which the shoulder-hanging band is similarly removed.

FIG. 9 is a lower side view for showing the saddle.

FIG. 10 is a side elevational view for showing the saddle.

FIG. 11 is a perspective view for showing a structure for fixing a thigh-abutting unit against the upper end of a shaft.

FIG. 12 is a perspective view for showing a structure for fixing a thigh-abutting unit against the upper end of a shaft.

FIG. 13 is a perspective view for showing a state in which a knee joint part formed at the midway portion of the shaft.

FIG. 14 is an outer appearance view for showing the lower end of the shaft.

FIG. 15 is an outer appearance view for showing the lower end of the shaft.

FIG. 16 is a sectional view taken along line A-A of FIG. 15.

FIG. 17 is a front elevational view for showing a state just before wearing the hucklebone supporting-type artificial leg of the present invention.

### PREFERRED EMBODIMENT OF THE INVENTION

Referring now to the drawings, the preferred embodiment of the present invention will be described as follows.

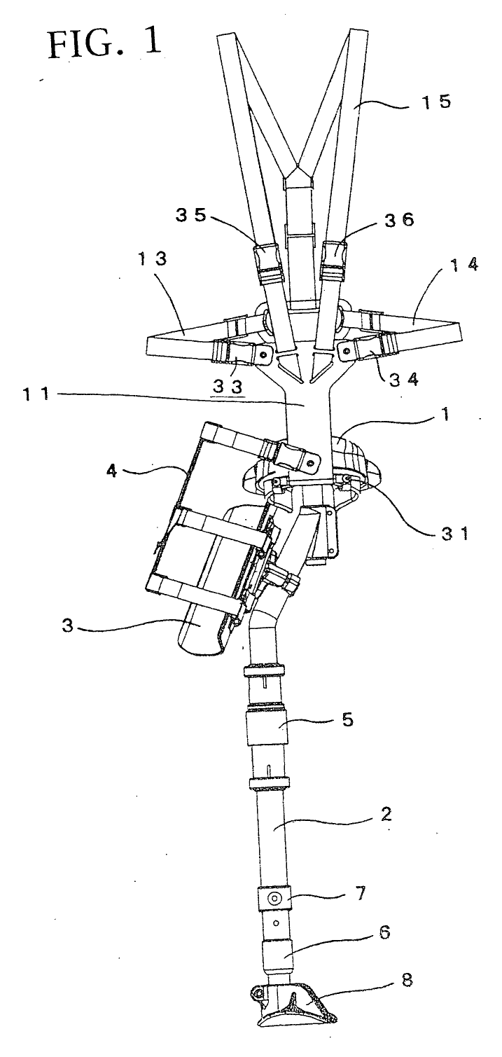

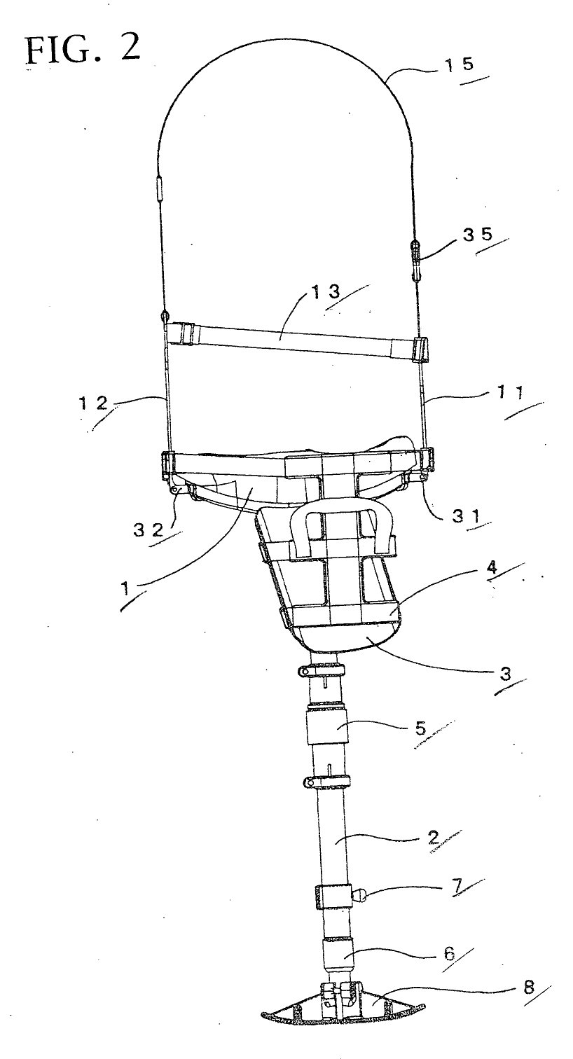

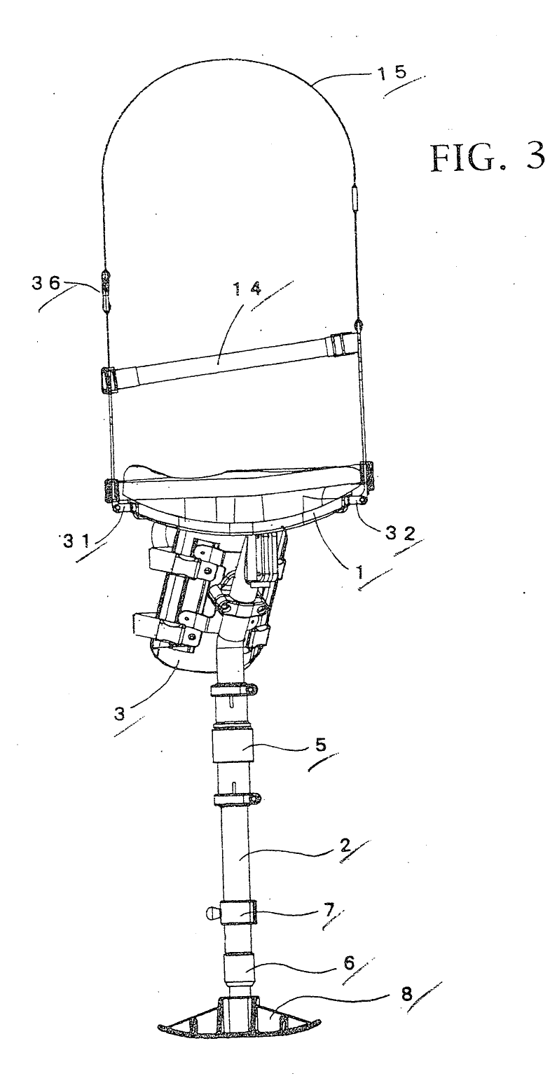

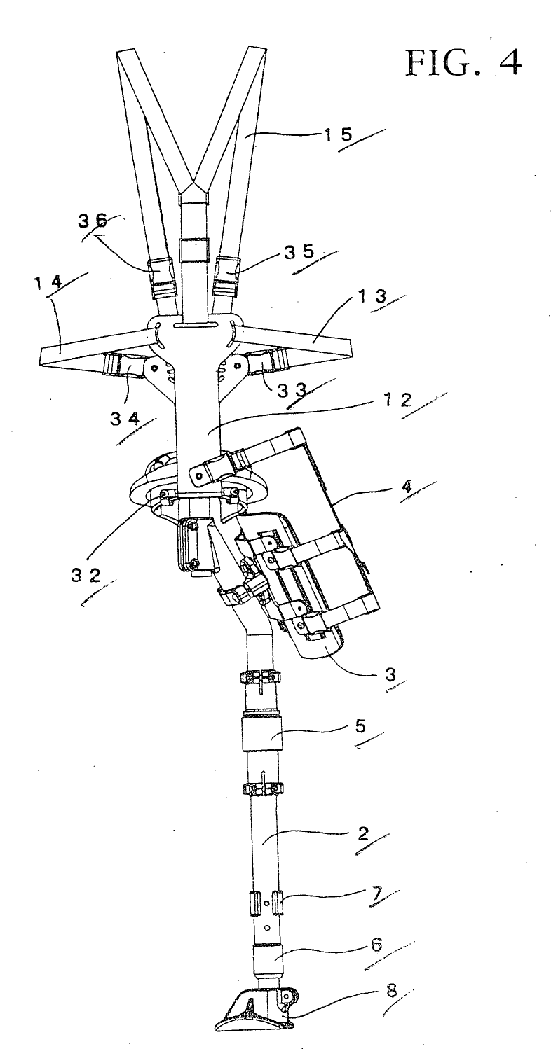

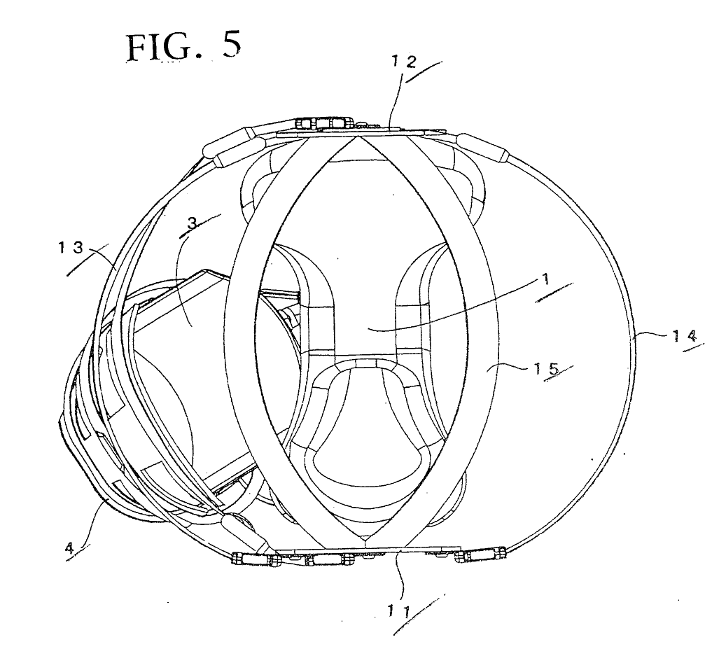

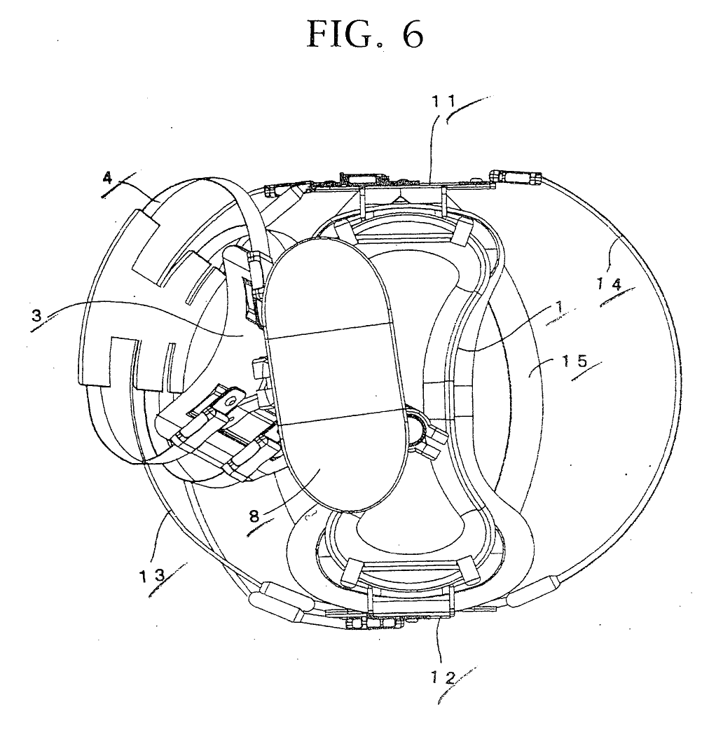

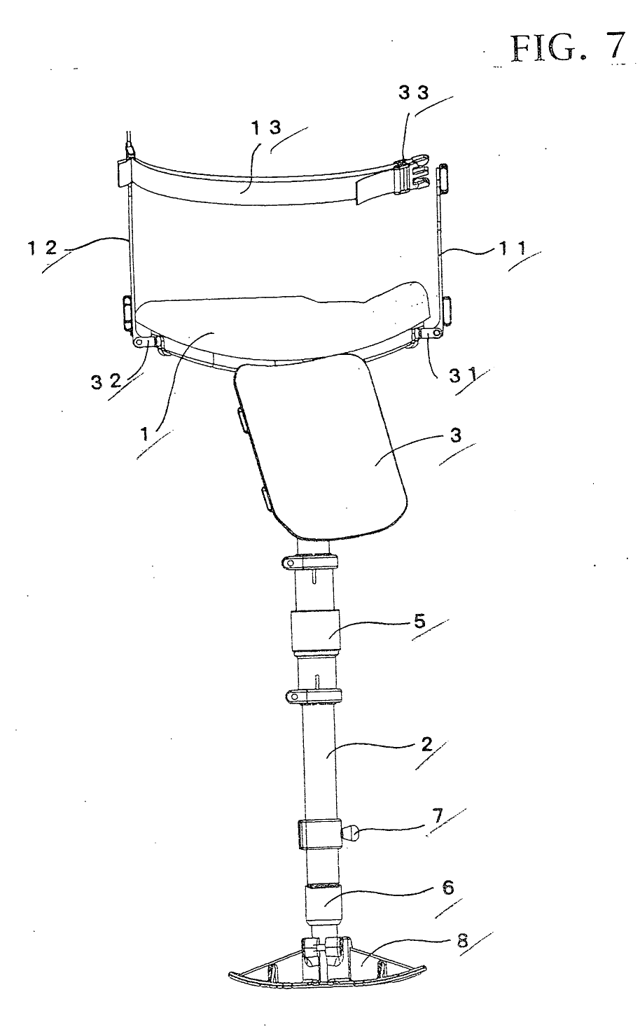

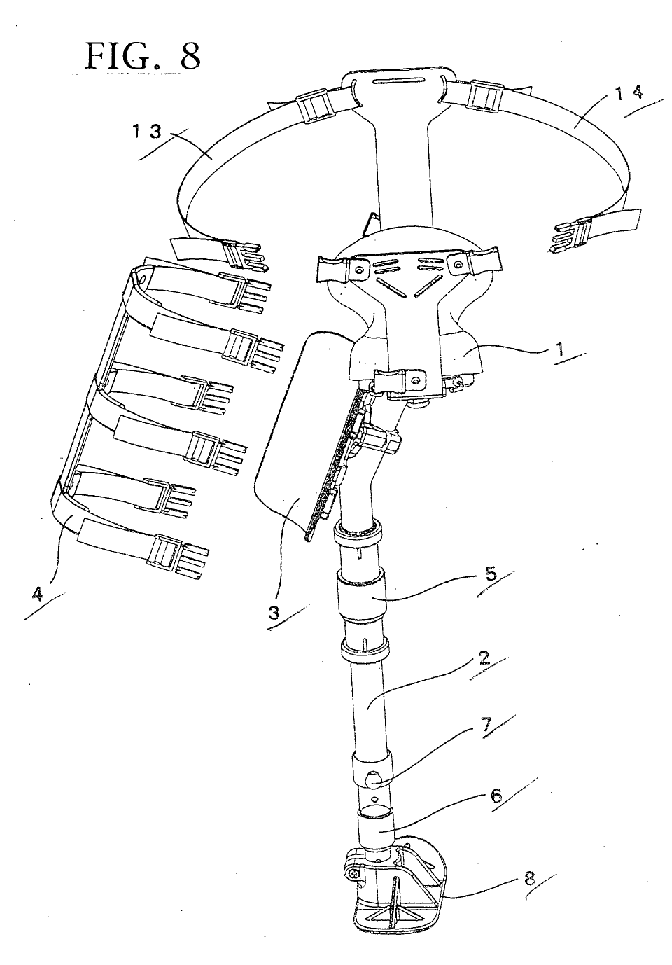

FIGS. 1 to 6 are views for showing a state in which the shoulder hanging band is connected to the hucklebone supporting-type artificial leg of the present invention. FIG. 1 is a front elevational view. FIG. 2 is a left side elevational view. FIG. 3 is a right side elevational view. FIG. 4 is a rear view. FIG. 5 is a top plan view. FIG. 6 is a bottom view. FIG. 7 is a left side elevational view for showing a state in which a shoulder-hanging band and a fastening band for fixing the thigh are removed from FIG. 2. FIG. 8 is a perspective view for showing a state in which a shoulder-hanging band is removed, and a waist belt and a fastening band for fixing the thigh are removed.

As shown in these figures, a shaft 2 is connected to a substantial central part of the lower surface of a saddle 1. At a location near the upper end of the shaft 2, the lower side of the shaft 2 is offset from the center of the saddle 1 in a slant forward direction by an arrangement in which the side of the damaged or lost lower leg is bent in a slant forward direction from a substantial central part of the lower portion of the saddle 1. A thigh-abutting unit 3 having the concave surface shape is fixed to the portion of the shaft 2 bent near the upper end in a slant forward and downward direction. Fastening bands 4 for use in pressing against the thigh are connected above the thigh-abutting unit 3. A collar 5 is fitted to a substantial midway position of the shaft 2 in such a way that the collar can be moved up and down and a knee joint to be described later is stored inside the collar.

A threaded collar 6 is threadably attached to the lower part of the shaft 2 below the collar 5. A part higher than the collar 6 is constituted into a double-shaft structure where two pipes of different diameter, some passing-through holes are formed at the part in an axial specified spacing and then a clip pin 7 is removably inserted into one of the passing-through holes. Loosening the collar 6 and pulling-off the clip pin 7 enable a length of the shaft 2 to be adjusted in a stepwise manner. A foot part 8 having a shape elongated in a forward or rearward direction is fixed to the lower end of the shaft 2. The foot part 8 has a shape in which both ends in a forward or rearward direction are warped in an upward direction, and the foot part 8 is fixed in a state wherein the center in the width direction is offset more inside than the center of the shaft 2.

In addition, a front belt supporting unit 11 is attached to the front end of the saddle 1 by a pair of right and left bracket fittings 31 in such a way that it may be turned in an upward or downward direction. Similarly, a rear belt supporting unit 12 is attached to the rear end of the saddle 1 by a pair of right and left bracket fittings 32 in such a way that it may be turned in an upward or downward direction. Each of the pair of right and left waist belts 13, 14 wound around the waist portion of the artificial leg wearing person is connected to the upper ends of these front belt supporting unit 11 and rear belt supporting unit 12, respectively. These waist belts 13, 14 can be engaged or disengaged and their adjustment in fastening operation can be performed through buckles 33, 34 arranged at the end portions of the waist belts. Further, the shoulder hanging bands 15 are connected to the upper ends of each of the front belt supporting unit 11 and the rear belt-supporting unit 12, respectively, and then applied to both shoulders of the artificial leg wearing person. The shoulder hanging bands 15 can be engaged or disengaged and their fastening adjustment can be carried out through the buckles 35, 36 arranged at the midway part of the shoulder hanging bands 15.

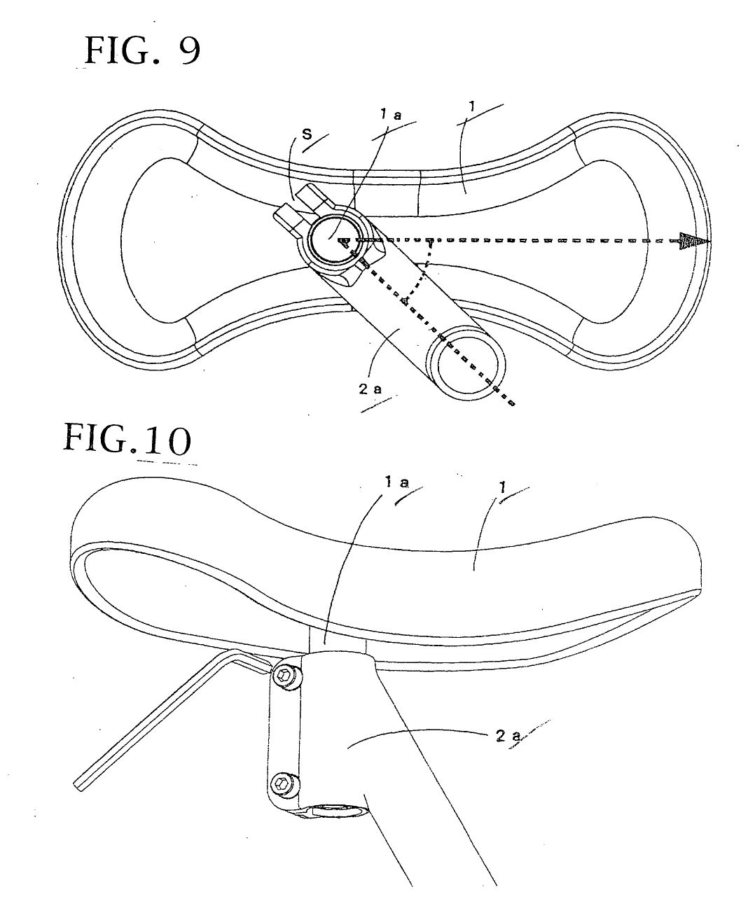

FIGS. 9 and 10 illustrate a connected portion between the saddle 1 and the shaft 2, wherein FIG. 9 is a bottom view for showing a portion of the saddle 1 and FIG. 10 is a side elevational view. As shown in these figures, the upper end 2a of the shaft 2 bent and formed in a slant crank shape is fitted to a saddle shaft 1a protruded at the lower surface of the saddle 1. Two bolts passing through the portion at a slit S formed at the side surface of the upper end 2a are fastened to cause the upper end 2a to be fixed to the saddle shaft la. The upper end 2a is swung in a lateral direction under a state in which the two bolts are being loosened and then directed toward the slant forward direction at the damaged or lost lower leg. It is preferable that the swinging angle at this time is 45°.

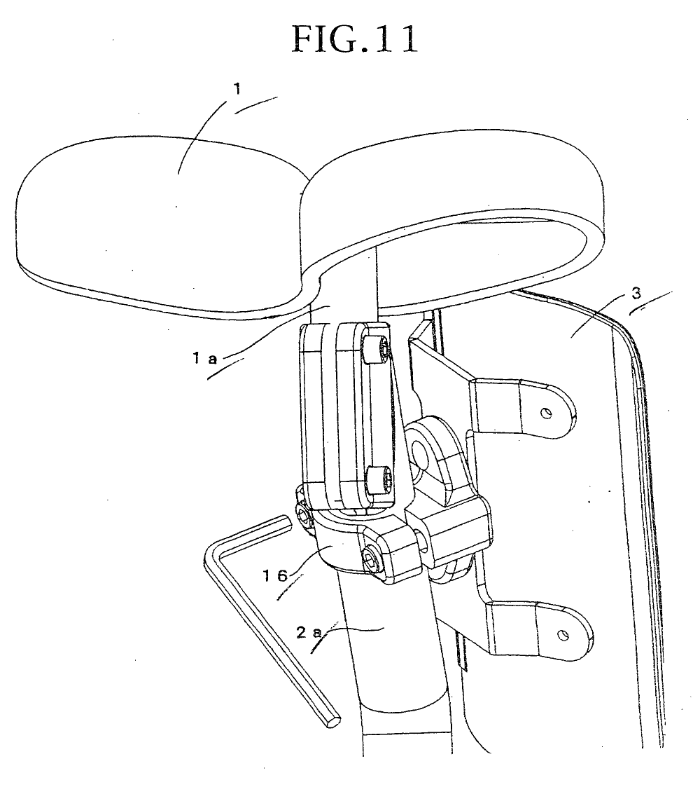

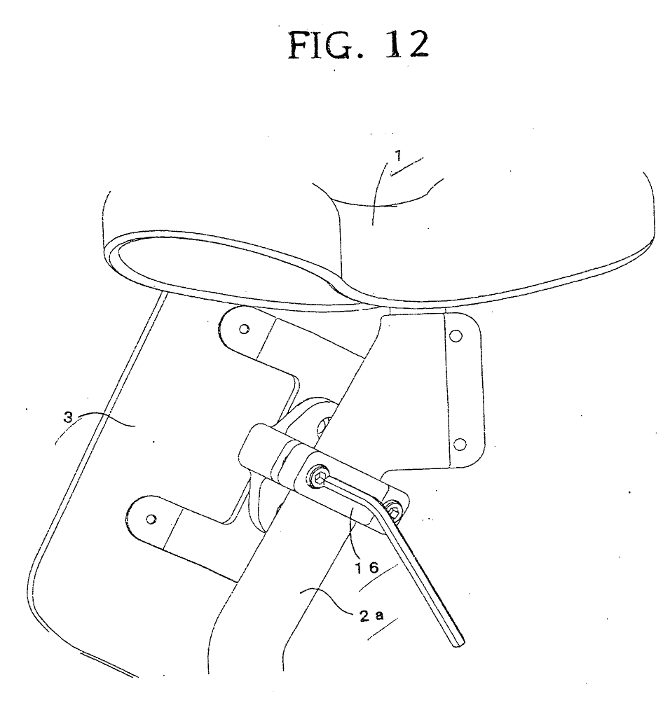

FIGS. 11 and 12 illustrate a structure for use in fixing the thigh-abutting unit 3 to the upper end 2a of the shaft 2. As shown, the saddle fitting 16 is abutted against the upper end 2a to fasten the two bolts, thereby the thigh-abutting unit 3 is fixed to the upper end 2a. The thigh-abutting unit 3 is rotatable in respect to the upper end 2a under a state in which the bolts are being loosened, and it becomes possible to direct the thigh-abutting unit 3 toward the slant forward and downward direction of the damaged and lost leg. In this case, the thigh abutting unit 3 is fixed to the upper end 2a toward the slant forward and downward direction to cause the thigh portion of the damaged or lost lower leg to be supported against the weight of the thigh itself and correspondingly the walking action becomes easy.

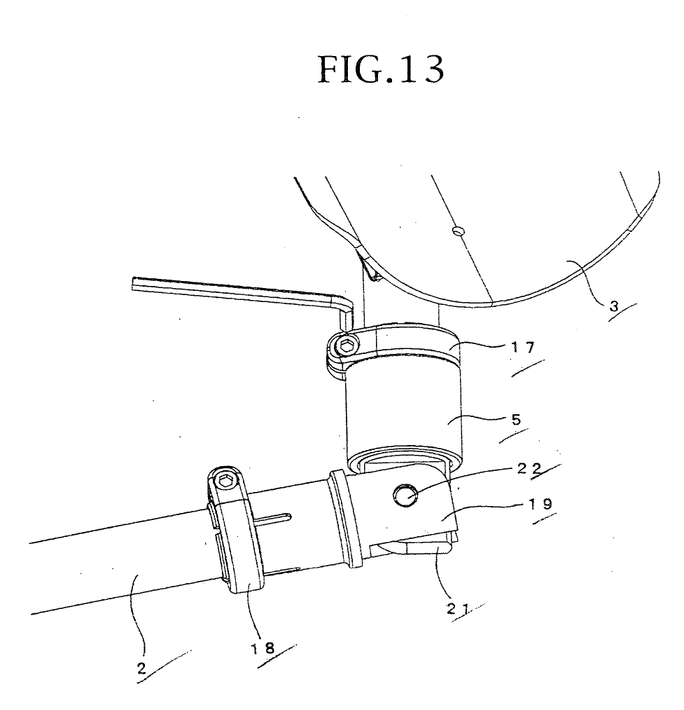

FIG. 13 is a perspective view for showing a state in which the knee joint part formed at the midway part of the shaft 2 is bent. The intermediate part of the shaft 2 is divided into an upper portion and a lower portion, wherein the set collars 17, 18 are fixed at the upper and lower portions and the collar 5 is rotatably fitted between them. A double-mountain shaped clevis 19 is formed at the lower end of the upper side of the divided segment, a protrusion 21 corresponding to the double-mountain shaped clevis 19 is formed at the lower end of the upper side of the divided segment, the pin 22 is inserted and passed under a state in which the double-mountain shaped clevis 19 and the protrusion 21 are fitted to each other and rotatably connected. The upper and lower portions of the shaft 2 are normally kept in a linear line-shaped rigid member state by descending the collar 5 to cover the knee joint part under a state in which the knee joint part is extended.

The knee joint constituted as described above enables the knee joint to be bent by bending the upper portion of the shaft 2 in a rearward direction after ascending the collar 5 when the artificial leg wearing person interrupts a walking action and sits on a chair or the like at the hip. When the artificial leg wearing person starts the walking action, the knee joint is extended to cause the collar 5 to be slid downward, the upper portion and the lower portion of the shaft 2 become a linear line state to enable the person to walk. Further, as shown in FIGS. 9 to 13, applying a bolt having hexagonal hole as a bolt to be used enables it to be decomposed or assembled with one hexagonal wrench.

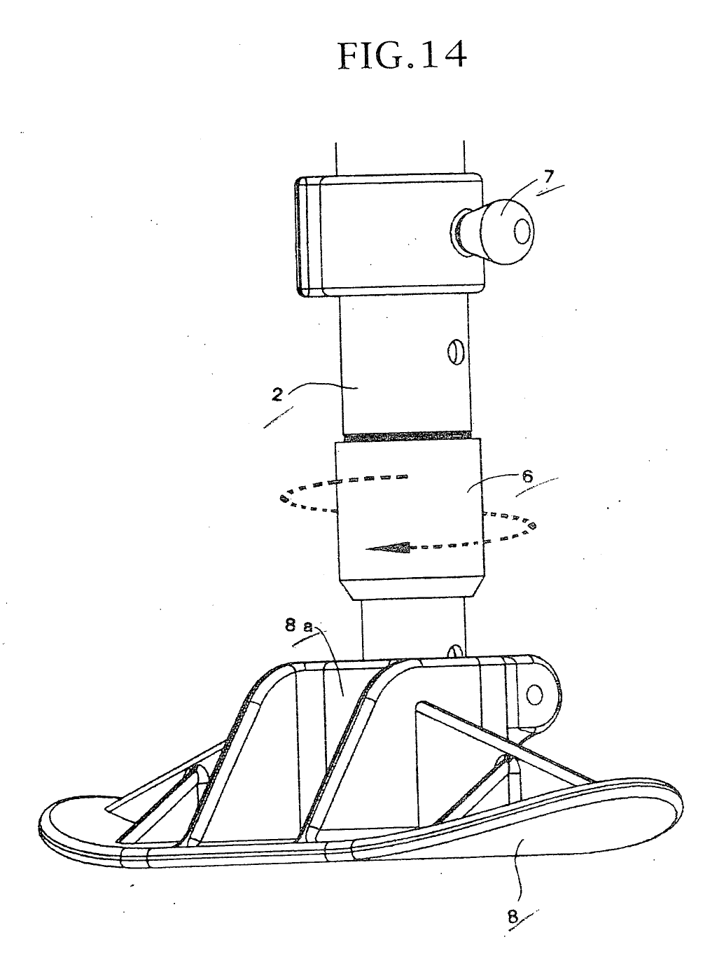

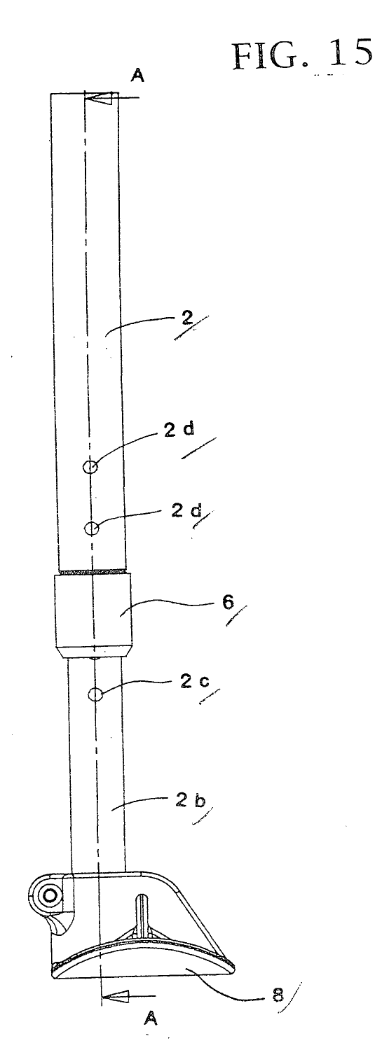

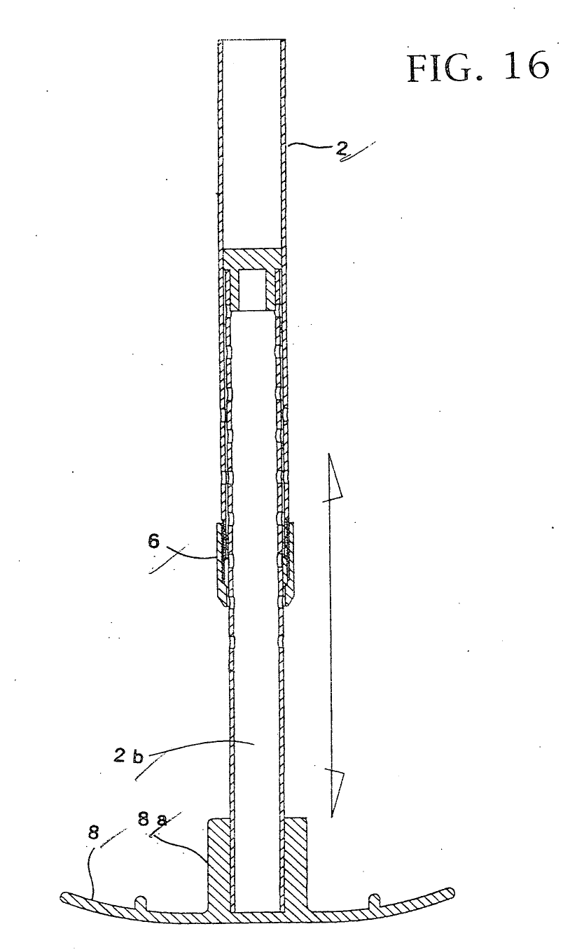

FIGS. 14 and 15 are outer appearance views for showing the lower end of the shaft 2, and FIG. 16 is a sectional view taken along line A-A of FIG. 15. As shown, the shaft 2 is made of a pipe material, and the middle-shaft 2b having a more fine size than that of the shaft 2 is inserted inside the lower end in such a way that it can be loaded or unloaded. Through-pass holes 2c, 2d are formed at the shaft 2 and the middle-shaft 2b in a diameter direction at a specified spacing. Adjusting an amount of fitting between the shaft 2 and the middle-shaft 2b and inserting a clip pin 7 into the through-pass holes 2c, 2d enable a length of the shaft 2 to be adjusted in a stepwise manner. The clip pin 7 is made such that a pin is fixed at the central part of the clip made of a f¶-shaped resilient member and it can be engaged or disengaged through one-finger touch operation. The outer circumference of the lower end portion of the shaft 2 is formed with a thread, the collar 6 having a thread is threadably fixed to the threaded portion and fastened there to cause the shaft 2 and the middle-shaft 2b to be connected to each other without producing any looseness.

In addition, the foot 8 is fixed by fastening a slit portion formed at the side surface of the hub portion 8a with a bolt under a state in which the lower end of the middle-shaft 2b is inserted into the hub portion 8b protruded at one side of the upper surface of the foot 8. Normally, although the center of the foot 8 is offset inside, it is also possible for the artificial leg wearing person to walk with the foot 8 being offset outside at the time of non-familiar with the walking just after wearing the artificial leg. When the upper end 2a of the shaft 2 is offset to either the right side or the left side of the leg, orientation of the foot 8 is correspondingly adjusted. In addition, also as shown in the bottom view of FIG. 6, it is preferable that the foot 8 is adjusted in such a way that its front end may face in a slant outside direction.

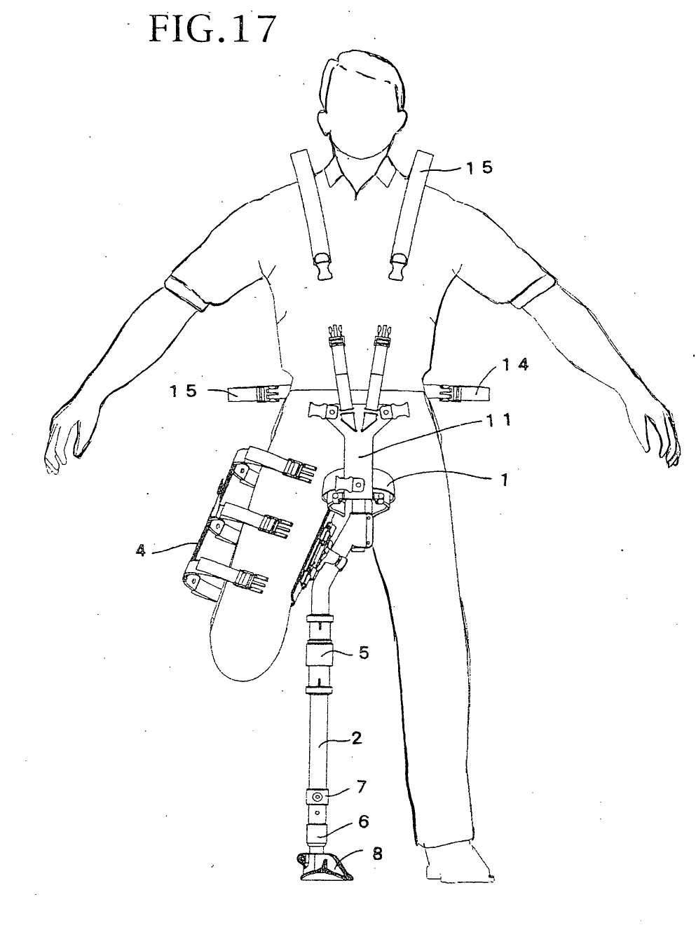

The hucklebone supporting-type artificial leg constituted as described above is installed as shown in FIG. 17. At first, the connected portion between the saddle 1 and the shaft 2 is loosened and the shaft 2 is fixed in compliance with the orientation of the damaged or lost lower leg of a wearing person. Similarly, the shaft 2 is also fixed with the orientation of the foot 8 is aligned. Next, the collar 6 is loosened and the clip pin 7 is pulled out of the hole, and then the length of the shaft 2 is adjusted in compliance with the normal side lower leg. In this case, the saddle 1 is abutted against the crotch and at the same time the thigh left at the damaged or lost lower leg is mounted on the thigh-abutting unit 3 and fixed under application of the fastening bands 4. At this time, both the front and rear buckles at the upper-most location of the fastening band 4 divided into three segments are connected to the lower end of each of the front belt supporting unit 11 and the rear belt supporting unit 12, respectively.

Then, the pair of right and left waist belts 13, 14 are wound around the waist part of the artificial leg wearing person, the buckles 33, 34 at their front ends are connected to the upper end of the front belt supporting unit 11 and at the same time their tension is adjusted. Further, the shoulder hanging bands 15 are applied to both shoulders of the artificial leg wearing person and connected to the front belt supporting unit 11 at the front portion by the buckles 35, 36 and at the same time their tension is adjusted. Further, the shoulder hanging bands 15 can also be eliminated in their wearing in the case that the saddle 1 is fixed positively by the waist belts 13, 14 and the fastening bands 4 at the thigh portion. Through these fastening operations, the artificial leg wearing work is completed and a wearing person can start immediately a walking action.

In addition, in the case that the artificial leg wearing person tries to ride on a vehicle or sit on a chair in the midway part of walking action, the person can descend the waist by shifting the collar 5 upward and bending the knee joint in a rearward direction after the physical weight is transferred to the normal lower leg side. Further, in the case that the person starts a walking action again, the person can perform the walking action while extending the knee joint and displacing the collar 5 in a downward direction. Further, in the case of the illustrated example, the front belt supporting unit 11 and the rear belt supporting unit 12 are fixed to the front and rear ends of the saddle 1, and the waist belts 13, 14 are connected to the upper ends of the supporting units. However, it is also possible to extend and arrange the front and rear ends of the saddle 1 in an upward direction and the waist belts 13, 14 are directly connected to the upper ends.

1. A hucklebone supporting-type artificial leg comprised of a saddle, a shaft suspended down at the lower surface of said saddle and having a substantial same length as that of a lower leg and a foot fixed to the lower end of said shaft, wherein the artificial leg comprising:
a front belt supporting unit attached at a front end of said saddle in such a way that it can be turned in a forward, upward and downward direction;
a rear belt supporting unit attached at a rear end of said saddle in such a way that it can be turned in a rearward, upward and downward direction; and
a waist belt connected to each of the upper ends of said front and rear belt supporting units and installed around the waist of an artificial leg wearing person in such a way that the belt can be fastened to the waist and engaged with or disengaged from the waist.

2. The hucklebone supporting-type artificial leg according to claim 1, wherein the same includes:
shoulder hanging bands having ends connected to each of upper ends of said front and rear belt supporting units and installed in such a way that they can be fastened to and removably set to the shoulders of the artificial leg wearing person.

3. The hucklebone supporting-type artificial leg according to claim 1, wherein
the upper portion of said shaft is bent to be offset from a connecting position at the center of said saddle in a slant forward direction of a damaged or lost lower leg.

4. The hucklebone supporting-type artificial leg according to claim 3, wherein
a connecting part between the upper end of said shaft and said saddle is of a fastening structure using a thread and an offset direction of the upper part of said shaft can be changed over between a right side and a left side.

5. The hucklebone supporting-type artificial leg according to claim 1, wherein
an intermediate position of a lower portion of said foot contacting with the ground and a position offset at one side in the right side or left side are applied as a connecting position with the lower end of said shaft; a connecting part between the lower end of said shaft and said foot is of a fastening structure using a thread; and an offset direction of said foot in respect to the lower end of said shaft can be changed over between a right side and a left side.

6. The hucklebone supporting-type artificial leg according to claim 1, wherein
the same includes a thigh abutting part attached to the upper portion of said shaft in a slant forward and downward direction and having a concave surface shape in which the thigh at the damaged or lost lower leg is fixed by a fastening band.

7. The hucklebone supporting-type artificial leg according to claim 1, wherein
a cylinder movable up and down is applied to cover an intermediate part of said shaft, the shaft in the cylinder at a position where said cylinder descends is divided into two upper and lower segments and connected by a hinge, and the upper portion of said shaft can be inclined in a rearward direction under a state in which said cylinder is ascended.

8. The hucklebone supporting-type artificial leg according to claim 1, wherein
an optional space at said shaft is of a double-shaft structure, a plurality of holes in a diameter direction are punched said double-shaft structure in an axial proper spacing, a pin is inserted into each of said holes in such a way that it can be inserted into or pulled out of the holes to enable the length of said shaft to be adjusted in a stepwise manner.
